(19) United States
(12) Patent Application Publication
(10) Pub. No.: US 2022/0039503 A1
(43) Pub. Date: Feb. 10, 2022

(54) PROTECTIVE HEADGEAR AND MULTIFUNCTIONAL CASUAL PROTECTIVE CLOTHING

(71) Applicant: ULTRA FOOTWEAR ACCELERATOR LLC, Waltham, MA (US)

(72) Inventor: Jian Deng, Waltham, MA (US)

(21) Appl. No.: 16/987,434

(22) Filed: Aug. 7, 2020

(51) Int. Cl. A42B 3/28; A41D 13/11; A41D 31/102; A41D 3/00; A41D 1/04; A41D 1/02; A41D 13/002; A61F 9/02

(52) U.S. Cl. CPC A42B 3/283; A41D 13/1153; A41D 13/1184; A41D 31/102; A41D 31/18; A41D 1/04; A41D 1/02; A41D 13/002; A61F 9/028; A41D 3/00

(57) ABSTRACT

The present invention relates to the technical field of clothing, and more particularly to a protective headgear and a multifunctional casual protective clothing. The protective headgear consists of a headgear body, an inner headgear, a goggle, breathing valves and a headgear zipper. The multifunctional casual protective clothing includes a piece of clothing and a protective headgear. A collar zipper is arranged on the collar of the clothing, and the collar zipper is connected with the headgear zipper of the headgear. By adopting the protective headgear, the multifunctional casual protective clothing of the present invention can avoid and prevent viruses, dust, smog, wind and snow; and after the protective headgear is removed, it can be used as ordinary casual clothing, with various functions and easy use, the protective headgear is suitable for casual clothing such as a coat, sportswear, down jacket, sweater, overcoat, jacket or leather jacket.

### FIELD OF THE PRESENT INVENTION

The present invention relates to the technical field of clothing, and more particularly to a protective headgear and a multifunctional casual protective clothing.

### DESCRIPTION OF RELATED ART

When an epidemic occurs, people should wear masks to prevent viruses when they go out. When a haze or sandstorm occurs, people should wear various masks and hats to prevent smog or dust when they go out. When a fire occurs, it is best to wear a gas mask to block the smog. When ice and snow whether occurs, people should wear various masks and hats to prevent the wind and snow when they go out.

However, most of the masks worn by people when they go out are disposable, which produces not only serious waste, but also does harms to the environment. Besides, hats can only protect the top of the people's heads, but not their eyes and faces. The existing special protective clothing includes a fire protective clothing, an protective clothing for industrial use, a protective clothing for medical use, a protective clothing for military use and a protective clothing for special groups, but these protective clothing are not suitable for daily wearing.

Therefore, it is necessary to invent a multifunctional casual protective clothing that can prevent and block viruses, dust, smog, wind and snow.

### SUMMARY OF THE PRESENT INVENTION

In order to overcome the shortcomings and deficiencies of the prior art, the purpose of the present invention is to provide a protective headgear and a multifunctional casual protective clothing that can avoid and block viruses, dust, smog, wind and snow.

The purpose of the present invention can be achieved by the following technical solution. The present invention provides a protective headgear including:

a headgear body made of a waterproof fabric, wherein a goggle is provided on the eyes area of the headgear body, and breathing valves are provided on outside of the mouth area of the headgear body;

an inner headgear made of an elastic fabric, wherein the top edge of the inner headgear is connected to the inside of the nose area of the headgear body;

a headgear zipper which is arranged on the back of the bottom of the headgear body and is used for linking with a collar zipper at the collar of the clothing.

A cape is extended from the bottom edge of the headgear body, and the cape is integrated with the headgear body.

A breathable mesh is arranged at a position of the inner headgear corresponding to the mouth area.

A brim is provided on the top of the goggle.

There are two breathing valves and the two breathing valves are symmetrically arranged on both sides of the mouth area of the headgear body.

The headgear body is made of an elastic waterproof and breathable fabric.

The goggle is an antifogging goggle.

The inner headgear is made of a soft and highly elastic Lycra fabric.

A multifunctional casual protective clothing, comprising a piece of clothing and the above-mentioned protective headgear, wherein a collar zipper is arranged at the collar of the clothing, and the collar zipper is connected with the headgear zipper of the protective headgear.

A cape is extended from the bottom edge of the headgear body, and the cape is integrated with the headgear body.

A breathable mesh is arranged at a position corresponding to the mouth area of the inner headgear.

A brim is provided on the top of the goggle.

There are two breathing valves and the two breathing valves are symmetrically arranged on both sides of the mouth area of the headgear body.

The headgear body is made of an elastic waterproof and breathable fabric.

The goggle is an antifogging goggle.

The inner headgear is made of a soft and highly elastic Lycra fabric.

A clothing zipper is provided on the front center of the clothing, and the clothing zipper is an easy-pulling zipper or a magnetic zipper.

The sleeves of the clothing are provided with elastic shrinking cuffs.

An elastic shrinking skirt is connected to the inner side of the waist area of the clothing, and two ends of the elastic shrinking skirt are connected by snap buttons.

The clothing is a coat, sportswear, down jacket, sweater, overcoat, jacket or leather jacket.

The beneficial effect of the present invention is as follow. By adopting an inner headgear, the protective headgear of the present invention can tighten the neck area and avoid and prevent viruses, dust, smog, wind and snow from entering the face, and by adopting a headgear zipper, the protective headgear can be connected with a collar zipper at the collar of the clothing, thus to combine into a multifunctional casual protective clothing, which can be detached and used conveniently.

By adopting the protective headgear, the multifunctional casual protective clothing of the present invention can avoid and block viruses, dust, smog, wind and snow, and after the protective headgear is removed, it can be used as an ordinary casual wear, with various functions and easy use, the protective headgear is suitable for casual clothing such as a coat, sportswear, down jacket, sweater, overcoat, jacket or leather jacket.

### BRIEF DESCRIPTION OF THE DRAWINGS

FIG. 1 is a perspective structural view of a protective headgear according to the present invention.

FIG. 2 is a perspective structural view of a multifunctional casual protective clothing according to the present invention.

FIG. 3 is a perspective exploded view of the multifunctional casual protective clothing according to the present invention.

FIG. 4 is a perspective structural view of the clothing according to the present invention.

The reference numbers in the drawings are as follows:
protective headgear 100, headgear body 10, goggle 11, breathable valve 12, inner headgear 13, headgear zipper 14, cape 15, breathable mesh 16, brim 17, multifunctional casual protective clothing 200, clothing 20, collar zipper 21, clothing zipper 22, elastic shrinking cuff 23, through-hole 24, elastic shrinking skirt 25, snap button 26.

### DETAILED DESCRIPTION OF THE EXEMPLARY EMBODIMENT

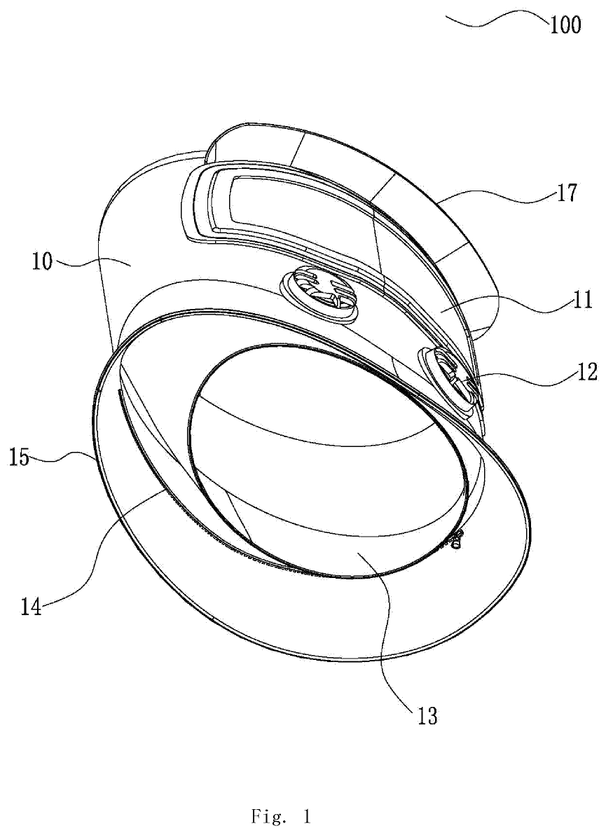

In order to facilitate the understanding for those skilled in the art, the present invention will be further described below with reference to the embodiments and FIGS. 1-4, and the contents mentioned in the embodiments are not intended to limit the present invention.

As shown in FIG. 1, a protective headgear 100 includes:

a headgear body 10 made of waterproof fabric, wherein a visible hole is provided on the eyes area of the headgear body 10, a goggle 11 is provided within the visible hole, and breathing valves 12 are arranged on the outside of the mouth area of the headgear body 10;

an inner headgear 13 made of an elastic fabric, wherein the top edge of the inner headgear 13 is connected to the inside of the nose area of the headgear body 10;

a headgear zipper 14 which is arranged on the back of the bottom of the headgear body 10 and used to connect with a collar zipper at the collar of the clothing.

Specifically, the top edge of the inner headgear 13 is sewn to the inner side of the nose area of the headgear body 10.

By adopting the inner headgear, the protective headgear of the present invention can tighten the neck area, and thus can prevent and block viruses, dust, smog, wind and snow, etc. from entering people's face. By adopting the headgear zipper, the protective headgear can be connected with the collar zipper at the collar of the clothing, thus to combine into a multifunctional casual protective clothing 200, which can be detached and used conveniently.

As shown in FIG. 1, a cape 15 is extended from the bottom edge of the headgear body 10, and the cape 15 is integrated with the headgear body 10. The cape 15 can surround people's neck to prevent viruses, dust, smog, wind and snow from entering people's face.

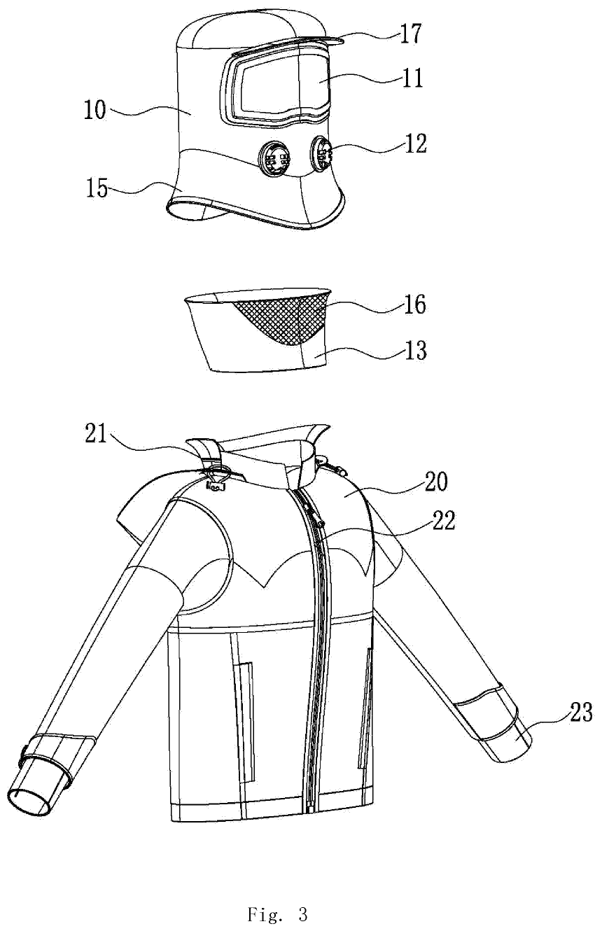

As shown in FIG. 3, a breathable mesh 16 is arranged on a position of the inner headgear 13 corresponding to the mouth area. The breathable mesh 16 can improve the smoothness of breathing.

As shown in FIG. 1, a brim 17 is provided on the top of he goggle 11, which can block sunlight and rain.

Specifically, there are two breathing valves 12, and both breathing valves 12 are symmetrically arranged on both sides of the mouth area of the headgear body 10. The breathing valves 12 are used to avoid inhaling viruses, dust, and smog when inhaling, and to exhale as much hot air as possible to avoid stuffy feeling and fogging the lens during wearing.

Specifically, the headgear body 10 is made of an elastic waterproof and breathable fabric. The material of the headgear body is an elastic waterproof and breathable fabric, whose breathability can avoid the stuffy feeling during wearing, and elasticity can maintain a certain sense of comfort during wearing. The shape is configured to be loose in a whole according to the head shape, and the brim is provided to maintain a sense of leisure.

Specifically, the goggle 11 is an antifogging goggle. The goggle 11 includes a frame and a lens. The frame is made of injection TPU material, and the lens is made of injection PC material. The lens is designed to be detachable and non-detachable, and is designed as an antifogging lens to prevent hot air fogging lens surface, which can be made in different colors.

Specifically, the inner headgear 13 is made of a soft and highly elastic Lycra fabric. The material of the inner headgear 13 is a soft and highly elastic Lycra fabric, which provides a good skin feel. The inner headgear 13 is configured to extend from the middle of the nasal bridge to the bottom of the neck, and it is designed to fit people's face and neck according to the human face and the curvature of the neck, thus to prevent viruses, dust, smog, wind and snow from entering people's face as much as possible.

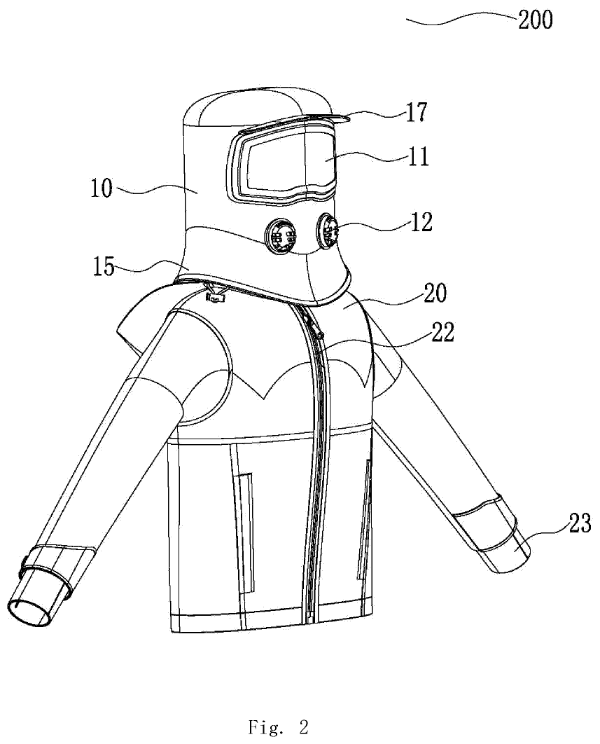

As shown in FIG. 2 to FIG. 3, a multifunctional casual protective clothing 200 includes a piece of clothing 20 and the above-mentioned protective headgear 100. A collar zipper 21 is arranged on the collar of the clothing 20, and the collar zipper 21 is connected with the headgear zipper 14 of the headgear bodyl0. By adopting the protective headgear 100, the multifunctional casual protective clothing 200 of the present invention can prevent and block viruses, dust, smog, wind and snow; and after the protective headgear 100 is removed, it can be used as ordinary casual clothing, with various functions and easy use, the protective headgear is suitable for casual clothing such as a coat, sportswear, down jacket, sweater, overcoat, jacket or leather jacket.

An opening is arranged on the front center of the clothing 20, and a piece of clothing zipper 22 is arranged on the opening. The clothing zipper 22 is an easy-pulling zipper or a magnetic zipper, and an ordinary zipper can also be used at the opening. The easy-pulling zipper or magnetic zipper is used in present invention, which is a humanized design and is easy to put on and take off. The easy-pulling zipper used in the present invention is a zipper that is convenient for taking off from double-head disclosed in a Chinese invention patent application No. 201710833913.5. The magnetic zipper used in the present invention is a magnetic tail-stop zipper disclosed in the Chinese invention patent application No. 201910912579.1.

As shown in FIG. 2 to FIG. 4, the sleeves of the clothing 20 are provided with elastic shrinking cuffs 23. Specifically, the elastic shrinking cuff 23 is provided with a through-hole 24 for a thumb to pass through. The elastic shrinking cuff 23 is made of an elastic and comfortable skin-fitting fabric which is close to the skin without a sense of restraint, thus to prevent viruses, dust, smog, and wind from entering the body through the cuff.

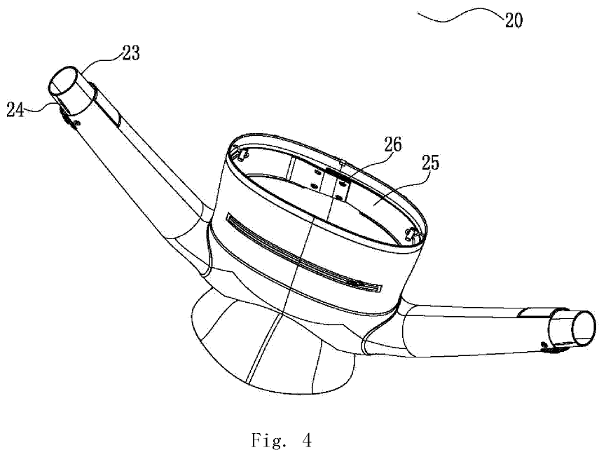

As shown in FIG. 4, an elastic shrinking skirt 25 is connected to the inner side of the waist area of the clothing 20, and both ends of the elastic shrinking skirt 25 are connected by snap buttons 26. The two ends of the elastic shrinking skirt 25 can also be connected by velcro tapes. The elastic shrinking skirt 25 is made of an elastic shrinking fabric, which is tightly attached to the waist and hip without a sense of restraint, thus to prevent viruses, dust, smog, and wind entering the body from below the waist and hip of the clothing. Specifically, the top edge of the elastic shrinking skirt 25 is sewn to the inner side of the waist of the clothing.

The clothing 20 of the present invention is a jacket, sportswear, down jacket, sweater, overcoat, jacket or leather jacket. The above-mentioned clothing may be long-sleeved or short-sleeved, as long as the collar zipper 21 can be arranged at the collar of the clothing.

By adopting the elastic shrinking cuffs attached to the wrists and the elastic shrinking skirt attached to the waist and hip, the clothing of the present invention can avoid and prevent viruses, dust, smog, wind and snow, etc. entering the body from the wrists, waist and hip, thus to form a protective clothing. By adopting the inner headgear to tighten the neck area, the protective headgear can avoid and prevent viruses, dust, smog, wind and snow, etc. from entering the face. The protective clothing and the protective headgear are combined into a whole to form an integrated multifunctional casual protective clothing capable of avoiding and preventing viruses, dust, smog, wind and snow, etc. from entering people's body and face.

The above mentioned embodiments are preferred implementation schemes of the present invention, in addition, the present: invention can be implemented in other ways. Any obvious replacement without departing from the concept of the present invention falls within. the protection scope of the present invention.

What is claimed is:

1. A protective headgear, comprising
a headgear body made of a waterproof fabric, wherein a goggle is provided on the eyes area of the headgear body, and breathing valves are provided on outside of the mouth area of the headgear body;
an inner headgear made of an elastic fabric, wherein the top edge of the inner headgear is connected to the inside of the nose area of the headgear body;
a headgear zipper which is arranged on the back of the bottom of the headgear body and is used for linking with a collar zipper at the collar of the clothing.

2. The protective headgear according to claim 1, wherein a cape is extended from the bottom edge of the headgear body, and the cape is integrated with the headgear body.

3. The protective headgear according to claim 1, wherein a breathable mesh is arranged at a position of the inner headgear corresponding to the mouth area.

4. The protective headgear according to claim 1, wherein a brim is provided on the top of the goggle.

5. The protective headgear according to claim 1, wherein there are two breathing valves and the two breathing valves are symmetrically arranged on both sides of the mouth area of the headgear body.

6. The protective headgear according to claim 1, wherein the headgear body is made of an elastic waterproof and breathable fabric.

7. The protective headgear according to claim 1, wherein the goggle is an antifogging goggle.

8. The protective headgear according to claim 1, wherein the inner headgear is made of a soft and highly elastic Lycra fabric.

9. A multifunctional casual protective clothing, comprising a piece of clothing and the protective headgear according to claim 1, wherein a collar zipper is arranged at the collar of the clothing, and the collar zipper is connected with the headgear zipper of the protective headgear.

10. The multifunctional casual protective clothing according to claim 9, wherein a cape is extended from the bottom edge of the headgear body, and the cape is integrated with the headgear body.

11. The multifunctional casual protective clothing according to claim 9, wherein a breathable mesh is arranged at a position corresponding to the mouth area of the inner headgear.

12. The multifunctional casual protective clothing according to claim 9, wherein a brim is provided on the top of the goggle.

13. The multifunctional casual protective clothing according to claim 9, wherein there are two breathing valves and the two breathing valves are symmetrically arranged on both sides of the mouth area of the headgear body.

14. The multifunctional casual protective clothing according to claim 9, wherein the headgear body is made of an elastic waterproof and breathable fabric.

15. The multifunctional casual protective clothing according to claim 9, wherein the goggle is an antifogging goggle.

16. The multifunctional casual protective clothing according to claim 9, wherein the inner headgear is made of a soft and highly elastic Lycra fabric.

17. The multifunctional casual protective clothing according to claim 9, wherein a clothing zipper is provided on the front center of the clothing, and the clothing zipper is an easy-pulling zipper or a magnetic zipper.

18. The multifunctional casual protective clothing according to claim 9, wherein the sleeves of the clothing are provided with elastic shrinking cuffs.

19. The multifunctional casual protective clothing according to claim 9, wherein an elastic shrinking skirt is connected to the inner side of the waist area of the clothing, and two ends of the elastic shrinking skirt are connected by snap buttons.

20. The multifunctional casual protective clothing according to claim 9, wherein the clothing is a coat, sportswear, down jacket, sweater, overcoat, jacket or leather jacket.
